(19) United States
(12) Patent Application Publication
(10) Pub. No.: US 2005/0129750 A1
(43) Pub. Date: Jun. 16, 2005

(54) Process for producing liposome suspension and product containing liposome suspension produced thereby

(76) Inventors: Yu-Fang Hu, Taipei (TW); Yao-Kun Huang, Taipei (TW); Chun-Chou Lin, Taipei (TW); Chi-Liang Kan, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/734,272

(22) Filed: Dec. 15, 2003

(51) Int. Cl. A61K 9/127; A61K 31/704; A61K 31/4745

(52) U.S. Cl. 424/450; 514/34; 514/283

(57) ABSTRACT

A process for the large scale production of a liposome suspension, in which three selected lipid compounds in a predetermined ratio are dissolved in an alcohol solvent to form a mixture, which, in turn, is directly admixed with an aqueous ammonium sulfate solution in a predetermined ratio. The resultant mixture is subjected to a pore-extrusion treatment, followed by dialyzing the pore-extruded mixture with a 5% to 15% sucrose aqueous solution, such that a liposome suspension containing liposome particles suspended in the liposome suspension is obtained. The thus obtained liposome suspension can be used to encapsulate a selected drug, in particular doxorubicin.

### BACKGROUND OF THE INVENTION

1. Field of the Invention

The present invention generally relates to a process for producing a liposome suspension and a liposome suspension produced thereby. The present invention also relates to a process for producing a product containing a liposome suspension and the product produced thereby, and particularly to a process for producing a liposome-encapsulated drug and the liposome-encapsulated drug produced thereby.

2. Description of Related Art

Since Dr. Alec D. Bangham disclosed the concept of using liposome as a delivery vehicle and the technique of efficiently manufacturing liposome in 1960 in England, liposome has become as an important target of research in the field of drug delivery. Liposomes are most frequently prepared from phospholipids, but other molecules of similar molecular shape and dimensions having both a hydrophobic and a hydrophilic moieties can be used.

Liposomes are microscopic vesicles, generally spherically shaped, formed from one or more lipid walls. The walls are prepared from lipid molecules, which have the tendency both to form bilayers and to minimize their surface area. The lipid molecules that make up a liposome have hydrophilic and lipophilic portions. Upon exposure to water, the lipid molecules form a bilayer membrane wherein the lipophilic portions of the lipid molecules in each layer are directed to the center of the membrane, and the opposing hydrophilic portions form the respective inner and outer surfaces of the bilayer membrane. Thus, each side of the membrane presents a hydrophilic surface while the interior of the membrane presents a lipophilic medium. The micelle liposome can have a particle size less than 20 nm. The micelle liposome can be used as a vector to deliver biologically active material to the body of human or animal in pharmaceutical manufacturing.

Liposomes can be classified into several categories based on their overall size and the nature of the lamellar structure. The categories include small unilamellar vesicles (SUV), multilamellar vesicles (MLV), large unilamellar vesicles (LUV), and oligolamellar vesicles (OLV). The range of the particle size of MLVs is about 0.4 μm to 1 μm. The range of the particle size of OLVs is about 0.1 μm to 1 μm. The range of the particle size of SUVs is about 20 nm to 100 nm. The range of the particle size of LUVs is about 1000 nm (R.R.C. New, ed. Liposomes: a practical approach, 1990, Oxford).

Conventional processes for producing liposomes are, for example, hydration, ultrasonification, dialysis and dilution processes, reverse phase evaporation (U.S. Pat. No. 4,235,871), surfactant treatment, dehydration-rehydration or dry-reconstitution, freeze and thaw (U.S. Pat. No. 6,355,267), pore extrusion, high pressure homogenization and so on (A. D. Bangham, M. M. Standish, J. C. Watkins, J. Mol. Biol., 13,238-252,1965; R. R. C. New, ed. Liposomes: a practical approach, 1990, Oxford; G. Gregoriadis, ed. Liposome Technology, vol. 1, 1993; C. R. C., H. Talsma and D. J. A. Crommelin, Pharmaceutical Technology, 96-106, 1992). The pore extrusion process and the high pressure homogenization process are used for large scale production of liposomes and have industrial potential. Based on economic considerations, the pore extrusion process is a better choice.

U.S. Pat. No. 4,737,323 discloses a method for using the pore extrusion method to produce a liposome suspension. The particle size of the liposome is uniform and has an average size less than about 0.4 μm. The method comprises providing a suspension of heterogeneous-size liposomes containing a substantial portion with sizes greater than 1.0 μm, and passing the suspension under pressure through an asymmetric ceramic filter whose inner-surface pore size is greater than the desired average liposome size and no greater than about 1.0 μm. According to the method, an organic solvent, e.g. chloroform in example 1, is used to dissolve lipids and then the organic solvent is removed by rotary evaporation under reduced pressure such that a dried thin film of lipids is formed. An aqueous medium is added continuously to the dried thin film of lipids, and the lipids are allowed to swell such that liposomes are formed.

Steven Lehigh's U.S. Pat. Nos. 5,004,611 and 5,053,217 disclose a method of making an aqueous dispersion of liposome that consists of a biologically active compound. The method consists essentially of mixing a pre-liposome composition comprising a uniform mixture of: (a) at least one bilayer forming membrane lipid, and (b) at least one non-aqueous liquid consisting essentially of a water-miscible organic liquid that is a solvent for the lipid. The proportion by weight of (a) to (b) is from 40:1 to 1:20.

Sufficient water is added to the pre-liposome such that liposomes are spontaneously formed. A biologically active compound is also added prior to, simultaneously or subsequent to the water and is present in a sufficient amount such that a biologically effective dosage of the biologically active compound is associated with the liposome. According to example 1 in the two U.S. patents, the pre-liposome composition is prepared at 50° C. to 60° C. The component (a) (for example, lecithin) is dissolved in the component (b) (for example, ethanol). The water portion is then added in two parts. The first part consists of an appropriate amount of the biologically active compound (for example, in a glucose solution), and an amount of distilled water in the second part is required to make up the final formulation. The pre-liposome compositions are equilibrated under N₂. Following equilibration, the pre-liposome compositions are cooled to 25° C. The liposomes are then prepared by a two-stage addition of phosphate buffer vigorously hand-shaken such that an aqueous dispersion of liposome that consists of a biologically active compound is formed. However, the ethanol is contained in the aqueous dispersion of liposome in a specific amount.

Francis C. Szoka, Jr. in U.S. Pat. Nos. 5,077,057, 5,277,914 and 5,549,910 discloses a method for preparing a lipid suspension of defined particle size encapsulating a useful compound that has poor water, alcohol or halogenated hydrocarbon solubility. The poorly-soluble compound and an encapsulating amount of a suitable lipid are dissolved in an aprotic solvent such as DMSO, optionally containing a solubilizing amount of a lower alcohol (for example, ethanol), and then extruded or injected into a stirred or mixed aqueous solution. The resulting liposomal suspension may be dialyzed or otherwise concentrated, if desired. The extrusion means may be a syringe, perforated plate or tube or other appropriate device providing apertures of about 0.05 mm to about 5 mm. In example 2 of the three U.S. Patents, doxorubicin is dissolved in DMSO and added to an ethanol solution containing egg phosphatidylglycerol (EPG): egg phosphatidylcholine (EPC): cholesterol (Chol) (7:3:6). Liposomes are formed by injecting the lipid-doxorubicin mixture into an aqueous phase consisting of 140 mM NaCl-10 mM Tris-HCl, pH 4.0, at 30° C. The liposome suspension is dialyzed and the liposome-encapsulated doxorubicin is separated from the nonencapsulated material by column chromatography. The resulting liposome particle diameter is 227 nm, and 41.2% of the doxorubicin is encapsulated in the liposome particles.

EP-A-560138 discloses a freeze-dried liposomal preparation containing a dihydro-pyridine compound that has a hydrophobic and low solubility character. U.S. Pat. No. 5,653,998 (R.O.C Pat. No. 359616) discloses that using short chain fatty acid (containing not greater than 10 carbon atoms) having a specific chemical formula or a salt of the fatty acid as a stabilizer causes the active compound having a hydrophobic and low solubility character to be encapsulated in the phospholipid membrane. The liposome can be a pharmaceutical preparation provided for parenteral delivery.

Martin C. Woodle et. al. in U.S. Pat. No. 5,013,556 disclose a liposome composition comprising liposomes composed of vesicle-forming lipids and between 1-20 mole percent of an amphipathic lipid derivatized with a polyethyleneglycol. One preferred amphipathic lipid is a phospholipid, such as phosphatidylethanolamine, derivatized with polyethyleneglycol. The liposomes can enhance circulation time in the bloodstream.

In example 10 of Francis J. Martin et. al. U.S. Pat. No. 5,213,804, a doxorubicin liposome is produced by using the liposome disclosed in U.S. Pat. No. 5,013,556. The selected lipid compositions in a selected mole ratio are dissolved in chloroform. Alpha-tocopherol (α-TC) in free base form is added in chloroform:methanol (2:1) solution. The lipid solution is dried to a thin lipid film, then hydrated with a warm solution of 125 mM ammonium sulfate containing 1 mM desferal. The lipid material is hydrated with 10 freeze/thaw cycles, using liquid nitrogen and a warm water bath. Liposome sizing is performed by extrusion. The extruded liposomes are then dialyzed against 5% glucose. A solution of doxorubicin is prepared and mixed with an equal volume of the dialyzed liposome preparation. The mixture is incubated at 60° C. in a water bath with shaking. Doxorubicin is encapsulated in the liposome.

Yechezkel Barenolz et. al. in U.S. Pat. No. 5,192,549 disclose a loading procedure for efficient loading of amphiphatic drugs into liposomes. The loading of the amphiphatic drug into liposomes is dependent on NH₄⁺ gradients between the internal and external aqueous phases of the liposome vesicles, in which gradients are created by forming liposomes in the ammonium solution, by subsequent ammonium removal from or dilution in the external aqueous phase of the liposomes. The liposomes create an outflow of neutral ammonia based on the created ammonium gradient, from internal to external aqueous phase, thus creating an active reverse, from outside to inside, pH gradient by accumulation of protons left behind by ammonia in the internal aqueous phase. An influx of deprotonated amphiphatic drug to liposomes based on the pH gradient replaces the departed ammonium.

The conventional methods for producing liposome have several disadvantages. Some methods use chloroform or other toxic organic solvents to dissolve the lipid so the method must remove the toxic organic solvents. Some methods have limiting in solvent injection so the methods can not operate in aseptic manipulation. The uniformity and the quality of the liposome can be influence by rotation speed, injection rate, solvent ratio, injection needle size and so on. The extrusion process in some methods can make the pressure over 150 to 250 psi, and the extrusion rate is very low (about 0.1 to 0.2 L/min). These disadvantages of the conventional methods will cause the manufacturing process to be time-consuming and prevent liposome from being manufactured in aseptic manipulation. The conventional methods cannot produce liposome in large scale.

### SUMMARY OF THE INVENTION

The main objective of the present invention is to provide a process for producing liposome suspension in large scale and preventing the necessity to remove toxic organic solvent.

To achieve the objective, a process for producing liposome suspension in accordance with the present invention comprises: (a) providing a pre-mixture to an alcohol solvent, (b) mixing the pre-mixture with an aqueous ammonium sulfate solution to form a mixture, (c) subjecting the mixture to a pore-extrusion treatment to form a pre-liposome suspension and (d) dialyzing the pre-liposome suspension with 5% to 15% sucrose aqueous solution.

In step (a), the pre-mixture comprises
(i) a phospholipid compound comprising 40%-70% of the pre-mixture and selected from the group consisting of lecithin, phosphatidylcholine (PC), phosphatidylethanolamine (PE), phosphatidylglycerol (PG), phosphatidylinositol, sphingomyelin (SM), phosphatidic acids, a di(C₁₂-C₁₈)acyl derivative of any of the foregoing and a combination of any of the foregoing;
(ii) a cholesterol comprising 10%-30% of the pre-mixture; and
(iii) a polyethyleneglycol (PEG)-derived compound comprising 15%-30% of the pre-mixture and selected from the group consisting of PEG-PE, methoxy-polyethyleneglycol (mPEG)-PE, a di(C₁₂-C₁₈)acyl derivative of either of the foregoing and a combination of any of the forgoing;
wherein the mole ratio of the alcohol solvent to the total amount of the compounds (i), (ii) and (iii) is greater than 5:1.

In step (b), the pre-mixture obtained in step (a) is mixed with an aqueous ammonium sulfate solution to form a mixture and the ratio of the amount of the pre-mixture obtained in step (a) to the aqueous ammonium sulfate solution is 1:2˜10 (v/v).

In step (c), the mixture obtained in step (b) is subjected to a pore-extrusion treatment and formed a pre-liposome suspension.

In step (d), the pre-liposome suspension obtained in step (c) is dialyzing with a 5% to 15% sucrose aqueous solution such that a liposome suspension contained liposome particles suspended therein is obtained.

The liposome suspension produced by the present invention can use to encapsulate an amphipathic drug. The other objective of the present invention is to provide a process for producing a liposome-encapsulated drug comprising:
mixing a selected drug and a liposome suspension produced by the foregoing process to produce a liposome-encapsulated drug contained the selected drug in the liposome particles in the liposome suspension. The selected drug is selected from the group consisting of an anthracycline antibiotic and a camptothecin anti-tumor drug. Preferably, the selected drug is selected from the group consisting of doxorubicin, daunorubicin, irinotecan and vinorelbine. More preferably, the selected drug is doxorubicin.

Further benefits and advantages of the present invention will become apparent after a careful reading of the detailed description with appropriate reference to the accompanying drawings.

### DETAILED DESCRIPTION OF THE INVENTION

A process for producing liposome suspension in accordance with the present invention comprises: (a) providing a pre-mixture to an alcohol solvent, (b) mixing the pre-mixture with an aqueous ammonium sulfate solution to form a mixture, (c) subjecting the mixture to a pore-extrusion treatment to form a pre-liposome suspension and (d) dialyzing the pre-liposome suspension with a 5% to 15% sucrose aqueous solution.

In step (a), the pre-mixture comprises
(i) a phospholipid compound comprising 40%-70% of the pre-mixture and selected from the group consisting of lecithin, phosphatidylcholine (PC), phosphatidylethanolamine (PE), phosphatidylglycerol (PG), phosphatidylinositol, sphingomyelin (SM), phosphatidic acids, a di(C12-C18)acyl derivative thereof and a combination of the foregoing;
(ii) a cholesterol comprising 10%-30% of the pre-mixture; and
(iii) a polyethyleneglycol (PEG)-derived compound comprising 15%-30% of the pre-mixture and selected from the group consisting of PEG-PE, methoxy-polyethyleneglycol (mPEG)-PE, a di(C₁₂-C₁₈)acyl derivative of any of the foregoing and a combination of the foregoing;
Wherein the mole ratio of the alcohol solvent to the total amount of compounds (i), (ii) and (iii) is more than 5:1.

In step (b), the pre-mixture obtained in step (a) is mixed with an aqueous ammonium sulfate solution to form a mixture and the ratio of the amount of the pre-mixture obtained in step (a) to the aqueous ammonium sulfate solution is 1:2˜10 (v/v).

In step (c), the mixture obtained in step (b) is subjected to a pore-extrusion treatment and forms a pre-liposome suspension.

In step (d), the pre-liposome suspension obtained in step (c) is dialyzed with a 5% to 15% sucrose aqueous solution such that a liposome suspension containing suspended liposome particles is obtained.

The alcohol solvent used in step (a) in the process is a non-toxic alcohol solvent and is water-soluble. Preferably, the alcohol solvent is selected from the group consisting of fatty alcohol, glycol, methanol, ethanol, i-propanol, ethylene glycol, propylene glycol and a combination of the foregoing alcohol solvents. In a preferred embodiment, the alcohol solvent used in step (a) is ethanol.

Preferably, the compound (i) used in step (a) is selected from the group consisting of PC, dilauroyl PC, dimyristoyl PC, dipalmitoyl PC, distearoyl phosphatidylcholine (DSPC), dioleoyl PC, dilinoleoyl PC, 1-palmitoyl-2-oleoyl PC and a combination of the foregoing compounds. In a preferred embodiment, the compound (i) used in step (a) is DSPC.

Preferably, the compound (iii) used in step (a) is selected from the group consisting of PEG-2000-PE, PEG-3000-PE, PEG-4000-PE, PEG-5000-PE, mPEG-2000-PE, mPEG-3000-PE, mPEG-4000-PE, mPEG-5000-PE, a di(C₁₂-C₁₈)acyl derivative of the foregoing compounds and a combination of any of the foregoing compounds.

More preferably, the compound (iii) used in step (a) is selected from the group consisting of PEG-2000-distearoyl phosphatidylethanolamine (DSPE), PEG-3000-DSPE, PEG-4000-DSPE, PEG-5000-DSPE and a di(C₁₂-C₁₈)acyl derivative of the foregoing.

The di(C₁₂-C₁₈)acyl derivative of compound (iii) used in step (a) can be but is not limited to 1,2-diacyl-SN-glycero-3-phosphatidyl ethanolamine-N-[methoxy(polyethylene glycol)-2000] and 1,2-diacyl-SN-glycero-3-phosphatidyl ethanolamine-N-[methoxy(polyethylene glycol)-3000]. The acyl is myristoyl, palmitoyl, stearoyl or oleoyl. In a preferred embodiment, the compound (iii) used in step (a) is PEG-2000-DSPE.

Preferably, the ratio of the amount of the alcohol solvent and the total amount of the compounds (i), (ii) and (iii) is 7˜10:1 (w/v). Preferably, the ratio of the amount of the pre-mixture obtained in step (a) to the aqueous ammonium sulfate solution is 1:4˜8 (v/v).

In a preferred embodiment, DSPC, cholesterol and PEG-2000-DSPE as components of liposome particles are dissolved in ethanol.

In a preferred embodiment, step (a) is carried out at 45° C. to 70° C. Preferably, the step (a) is carried out at 55° C. to 65° C. More preferably, the step (a) is carried out at 60° C.

In a preferred embodiment, DSPC, cholesterol and PEG-2000-DSPE are dissolved in ethanol and mixed well in a water bath at 60° C.

In the step (b) of the process, the lipid/organic solvent mixture is not like the convention process that uses a syringe to inject an small amounts of aqueous solution several times. The present process can directly add the aqueous ammonium sulfate solution to the pre-mixture obtained in step (a) or directly add the pre-mixture obtained in step (a) to the aqueous ammonium sulfate solution.

Preferably, step (b) is carried out at 45° C. to 70° C. More preferably, the step (b) is carried out at 55° C. to 65° C. Most preferably, the step (b) is carried out at 60° C. At this operation temperature, the lipid bilayer membrane structure of the liposome particle in the mixture is loose. The liposome particle will encapsulate small amounts of aqueous ammonium sulfate solution.

Preferably, the equivalent weight of the aqueous ammonium sulfate solution in step (b) is 0.2N to 0.8N. More preferably, the equivalent weight of the aqueous ammonium sulfate solution in step (b) is 0.4N to 0.6N.

Preferably, the pore-extrusion treatment in step (c) passes the mixture obtained in step (b) through a device with apertures of 0.05 μm to 0.45 μm.

A device to perform the pore-extrusion treatment in step (c) in accordance with the present invention may be a syringe providing apertures of about 0.05 μm to 0.45 μm, a filter containing a ceramic filtration membrane or a polycarbonate filtration membrane or a plate or tube with apertures.

Preferably, the pore-extrusion treatment in step (c) has two steps and first passes the mixture obtained in step (b) through a filter having large apertures and then through a filter having small apertures. In a preferred embodiment, the pore-extrusion treatment in step (c) passes the mixture obtained in step (b) through a filter having apertures of 0.1 μm and then a filter having apertures of 0.05 μm at 60° C.

Preferably, step (d) is carried out at room temperature. Under these conditions, the lipid bilayer of the liposome particle will become dense, and the aqueous ammonium sulfate solution will not escape from the inside of the liposome particle to the sucrose solution during dialyzation.

The liposome suspension obtained in step (d) can be using immediately or can be stored at a low temperature (for example, 5° C.), or the obtained liposome suspension is further lyophilized and stored at a low temperature (for example, 5° C.).

Compared with the conventional process, the process for producing liposome suspension in accordance with the present invention can be performed at a low pressure (about 40 to 140 psi), and the yield increases (about 2 to 10 L/minute). The present invention can provide a quick and economic process for producing liposome suspension in large quantities.

The liposome suspension produced by the present invention can be used in pharmaceuticals and cosmetics, more particularly for encapsulating a selected drug that can be but is not limit to anthracycline antibiotics and camptothecin anti-tumor drugs.

Thus, the present invention also provides a process for producing a liposome-encapsulated drug comprising:
mixing a selected drug and a liposome suspension produced by the foregoing process to produce a liposome-encapsulated drug containing the selected drug in the liposome particles in the liposome suspension.

Preferably, the selected drug is selected from the group consisting of doxorubicin, daunorubicin, irinotecan and vinorelbine.

Preferably, the selected drug and liposome suspension are mixed at 45° C. to 70° C. More preferably, the selected drug and liposome suspension are mixed at 55° C. to 65° C. Most preferably, the selected drug and liposome suspension are mixed at 60° C. At this temperature, the lipid bilayer of the liposome particle is loose and allows the selected drug to enter the liposome particles and bind with the ammonium sulfate. The temperature is decreased to room temperature, and the lipid bilayer of the liposome particle becomes more dense such that the selected drug is stably encapsulated in the liposome particles in the liposome suspension.

Preferably, a pharmaceutical acceptable additive is used when a drug is encapsulated. For example, the pharmaceutical acceptable additive is a cryoprotector, an antioxidant formed stabilizer, a pH-regulator, a dispersing agent or a combination of the foregoing. For example the cryoprotector may be polyol (glycerol), monosaccharide (glucose), disaccharide (sucrose, lactose, trehalose), a protein or an amino acid (histidine). The antioxidant formed stabilizer may be butylated hydroxyanisole (BHA), butylated hydroxytoluene (BHT), α-tocopherol or a salt of α-tocopherol, ascorbic acid or a salt or an ester of ascorbic acid, preferably is ascorbic acid and a salt thereof. The pH-regulator may be a buffer, an acid or a base (for example ascorbic acid and NaOH). The dispersing agent may be glycerol, mannitol or glucose.

In a preferred embodiment, the selected drug is doxorubicin that is encapsulated in the liposome suspension produced by the present invention and histidine as the stabilizer.

All of the documents or publications recited in the text are incorporated herein by reference.

Further details of this invention are illustrated in the following examples.

Materials

A. Aqueous Ammonium Sulfate Solution:

158 g ammonium sulfate (Showa Co., Japan) was added to injection water, and the volume was adjusted to 6 L. The solution was filtered with Posydine (Whatman Inc., Germany) thin membrane (142 mm, 0.22 μm) and stored at 6° C. Before using, the ammonium sulfate aqueous solution was adjusted at 60° C.

B. 9% (w/w) Sucrose Solution:

4500 g sucrose was dissolved in a suitable amount of injection water and diluted to 50 L, and then filtered with Posydine thin membrane (142 mm, 0.22 μm).

C. Histidine-Sucrose Solution:

12.6 g histidine monohydrate (Ajinomoto Co., Japan) was dissolved in 90 mL 9% sucrose solution, and 1N NaOH was used to adjust the range of the pH from 6.2 to 6.6.9% sucrose solution was added until the volume was 300 mL and mixed well. The solution was filtered with Posydine thin membrane (47 mm, 0.22 μm).

### EXAMPLE 1

### Preparation of Liposome Suspension

16.8 g of PEG-2000-DSPE (Genzyme Co., America), 27.4 g of cholesterol (NOF Co., Japan) and 38.2 g of DSPC(NOF Co., Japan) were added to 600 ml of ethanol in a glass container. The mixture was stirred at 60° C. and mixed well. While continuously stirring the mixture and maintaining the mixture at 60° C., 4 L of the aqueous ammonium sulfate solution was directly added to the mixture. At the temperature, the ethanol was almost evaporated. Then the mixture was subjected to a pore-extrusion treatment using a 1.5 L of filter (Advantec Toyo Kaisha, Ltd., Japan), and the pore-extrusion treatment comprised
(1) a filter having a first filtration membrane (142 mm, 0.1 μm) and filtering the mixture 10 times; and
(2) changing to a second filtration membrane (142 mm, 0.05 μm) and filtering the mixture more 10 times.

The extrusion pressure was controlled at 3 to 10 kg/cm² and the flow rate was about 2 to 10 L/min. 4500 mL of filtration solution was collected and then dialyzed with 30 L 9% (w/w) sucrose solution previously prepared in a 30 KD hollow fiber (A/G Technology, UFP-30-C-6A, 30,000 NM, 4800 cm²). The remnant ethanol was all removed by dialyzing. The volume of the collected solution was about 3000 mL, and the collected solution was a liposome suspension that did not contain ethanol.

Product Quality Analysis

A sample of the liposome suspension produced in example 1 was analyzed with a GC analyzer (Varian, Inc., America). The results showed that the sample of the liposome suspension contained no ethanol. A particle size analysis was performed on another sample with a particle size analyzer (Beckman coulter, Inc.). The results showed the average particle size in the liposome suspension in the sample to be 72.9 nm.

### EXAMPLE 2

### Preparation of Liposome Encapsulated Doxorubicin

3000 mL of liposome suspension produced in example 1 was added to a glass container containing 8000 mg doxorubicin HCl (red powder), and 200 mL histidine-sucrose solution previously prepared was continuously added. A mixture was formed and put in a 60° C. water bath and stirred for 30 minutes. The mixture was then cooled to about 35° C., diluted with 9% sucrose solution to 4 L and mixed well. A liposome-encapsulated doxorubicin was produced. The product was further packaged in sterile glass vials and manufactured an injection preparation containing 2.0 mg doxorubicin HCl/mL.

Product Quality Analysis

The color and luster of the liposome-encapsulated doxorubicin produced in example 2 was observed. The color of the injection preparation packaged in the sterile glass vials was reddish orange to red. A sample was analyzed with an HPLC analyzer (Waters co., America) and compared with the standard. The results showed that the retention time and elution profile of the doxorubicin HCl in the sample were the same as that of the standard.

The liposome-encapsulated doxorubicin produced in example 2 was analyzed to determine the amount of doxorubicin encapsulated. 99.61% of the doxorubicin was encapsulated in the liposome. A sample was analyzed to determine the particle size with a particle size analyzer (Beckman coulter, Inc.), and the results showed that the particle size of the liposome in the sample was 91.0 nm.

The liposome-encapsulated doxorubicin produced in example 2 was stored at 2 to 8° C. for 30 months. Testing of a stored sample showed that the stored sample was stable (table 1), and the particle size of the liposome had not apparently changed.

TABLE 1
Stability of the Injection Preparation of the Liposome-
encapsulated Doxorubicin in Example 2

| | Concentration (mg/mL) | Particle size (nm) |
|---|---|---|
| Time | 5° C. ± 3° C. | 5° C. ± 3° C. |
| Beginning | 1.99 ± 0.02 | 91.0 ± 22.2 |
| 1^{st} month | 1.97 ± 0.05 | 95.2 ± 24.1 |
| 12^{th} month | 1.95 ± 0.07 | 90.4 ± 18.3 |
| 24^{th} month | 1.96 ± 0.03 | 96.3 ± 23.1 |
| 30^{th} month | 1.98 ± 0.01 | 93.3 ± 26.6 |

Although the invention has been explained in relation to its preferred embodiment, many other possible modifications and variations can be made without departing from the spirit and scope of the invention as hereinafter claimed.

1. A process for producing a liposome suspension comprising:
(a) providing a pre-mixture to an alcohol solvent, wherein the pre-mixture comprises
(i) a phospholipid compound comprising 40%-70% of the pre-mixture and selected from the group consisting of lecithin, phosphatidylcholine (PC), phosphatidylethanolamine (PE), phosphatidylglycerol (PG), phosphatidylinositol, sphingomyelin (SM), phosphatidic acids, a di(C₁₂-C₁₈)acyl derivative of any of the foregoing and a combination of any of the foregoing;
(ii) a cholesterol comprising 10%-30% (w/w) of the pre-mixture; and
(iii) a polyethyleneglycol (PEG)-derived compound comprising 15%-30% (w/w) of the pre-mixture and selected from the group consisting of PEG-PE, methoxy-polyethyleneglycol (mPEG)-PE, a di(C₁₂-C₁₈)acyl derivative of either of the foregoing and a combination of any of the foregoing;
wherein the ratio of the alcohol solvent to the total amount of compounds (i), (ii) and (iii) is greater than 5:1;
(b) mixing the pre-mixture obtained in step (a) with an aqueous ammonium sulfate solution to form a mixture, wherein the ratio of the amount of the pre-mixture obtained in step (a) to the aqueous ammonium sulfate solution is 1:210 (v/v);
(c) subjecting the mixture obtained in step (b) to a pore-extrusion treatment and forming a pre-liposome suspension; and
(d) dialyzing the pre-liposome suspension obtained in step (c) with a 5% to 15% sucrose aqueous solution such that a liposome suspension containing liposome particles suspended in the liposome suspension is obtained.

2. The process as claimed in claim 1, wherein the alcohol solvent used in step (a) is selected from the group consisting of fatty alcohol, glycol, methanol, ethanol, i-propanol, ethylene glycol, propylene glycol and a combination of any of the foregoing alcohol solvents.

3. The process as claimed in claim 1, wherein the alcohol solvent used in step (a) is ethanol.

4. The process as claimed in claim 1, wherein the compound (i) used in step (a) is selected from the group consisting of PC, dilauroyl PC, dimyristoyl PC, dipalmitoyl PC, distearoyl phosphatidylcholine (DSPC), dioleoyl PC, dilinoleoyl PC, 1-palmitoyl-2-oleoyl PC and a combination of any of the foregoing compounds.

5. The process as claimed in claim 1, wherein the compound (i) used in step (a) is DSPC.

6. The process as claimed in claim 1, wherein the compound (iii) used in step (a) is selected from the group consisting of PEG-2000-PE, PEG-3000-PE, PEG-4000-PE, PEG-5000-PE, mPEG-2000-PE, mPEG-3000-PE, mPEG-4000-PE, mPEG-5000-PE, a di(C₁₂-C₁₈)acyl derivative of the foregoing compounds and a combination of any of the foregoing compounds.

7. The process as claimed in claim 1, wherein the compound (iii) used in step (a) is selected from the group consisting of PEG-2000-DSPE, PEG-3000-DSPE, PEG-4000-DSPE, PEG-5000-DSPE, 1,2-diacyl-SN-glycero-3-phosphatidyl ethanolamine-N-[methoxy(polyethylene glycol)-2000] and 1,2-diacyl-SN-glycero-3-phosphatidyl ethanolamine-N-[methoxy(polyethylene glycol)-3000], wherein the acyl is myristoyl, palmitoyl, stearoyl or oleoyl.

8. The process as claimed in claim 1, wherein the compound (iii) used in step (a) is PEG-2000-DSPE.

9. The process as claimed in claim 1, wherein the ratio of the amount of the alcohol solvent to the total amount of compounds (i), (ii) and (iii) is 7˜10:1 (w/v).

10. The process as claimed in claim 1, wherein the compound (i) is DSPC and the compound (iii) is PEG-2000-DSPE in step (a).

11. The process as claimed in claim 1, wherein step (a) is carried out at 45° C. to 70° C.

12. The process as claimed in claim 1, wherein step (a) is carried out at 55° C. to 65° C.

13. The process as claimed in claim 1, wherein step (a) is carried out at 60° C.

14. The process as claimed in claim 1, wherein step (b) is carried out at 45° C. to 70° C.

15. The process as claimed in claim 1, wherein step (b) is carried out at 55° C. to 65° C.

16. The process as claimed in claim 1, wherein step (b) is carried out at 60° C.

17. The process as claimed in claim 1, wherein the equivalent weight of the aqueous ammonium sulfate solution in step (b) is 0.2N to 0.8N.

18. The process as claimed in claim 1, wherein the equivalent weight of the aqueous ammonium sulfate solution in step (b) is 0.4N to 0.6N.

19. The process as claimed in claim 1, wherein the ratio of the amount of the pre-mixture obtained in step (a) to the aqueous ammonium sulfate solution is 1:4-8 (v/v).

20. The process as claimed in claim 1, wherein the pore-extrusion treatment in step (c) passes the mixture obtained in step (b) through a device having apertures of 0.05 μm to 0.45 μm.

21. The process as claimed in claim 20, wherein the device is selected from the group consisting of a syringe having apertures, a filter containing a ceramic filtration membrane or a polycarbonate filtration membrane and a plate or tube having apertures.

22. The process as claimed in claim 1, wherein the pore-extrusion treatment in step (c) is composed of two steps and first passes the mixture obtained in step (b) through a filter having large apertures and then through a filter having small apertures.

23. The process as claimed in claim 22, wherein the large apertures are 0.1 μm and the small apertures are 0.05 μm.

24. The process as claimed in claim 1, wherein step (d) is carried out at room temperature.

25. The process as claimed in claim 1, wherein the obtained liposome suspension is further lyophilized.

26. A process for producing a liposome-encapsulated drug comprising:
mixing a selected drug and a liposome suspension produced by the process as claimed in claim 1 to produce a liposome-encapsulated drug containing the selected drug in the liposome particles suspended in the liposome suspension.

27. The process for producing a liposome-encapsulated drug as claimed in claim 26, wherein the selected drug is selected from the group consisting of an anthracycline antibiotic and a camptothecin anti-tumor drug.

28. The process for producing a liposome-encapsulated drug as claimed in claim 27, wherein the selected drug is selected from the group consisting of doxorubicin, daunorubicin, irinotecan and vinorelbine.

29. The process for producing a liposome-encapsulated drug as claimed in claim 27, wherein the selected drug is doxorubicin.

30. The process for producing a liposome-encapsulated drug as claimed in claim 26, wherein the selected drug and the liposome suspension are mixed at 45° C. to 70° C. and then reduced to room temperature such that the selected drug is encapsulated in the liposome particles suspended in the liposome suspension.
